(19) United States
(12) Patent Application Publication
(10) Pub. No.: US 2021/0170091 A1
(43) Pub. Date: Jun. 10, 2021

(54) CANNULAS AND CANNULA ASSEMBLIES FOR HEMODIALYSIS

(71) Applicant: Carlos G. SANCHEZ, Miami Beach, FL (US)

(72) Inventor: Carlos G. SANCHEZ, Miami Beach, FL (US)

(21) Appl. No.: 16/707,788

(22) Filed: Dec. 9, 2019

(51) Int. Cl. A61M 1/36

(52) U.S. Cl. CPC A61M 1/3661; A61M 1/3655

(57) ABSTRACT

A cannula assembly comprises a cannula including a first portion, and a second portion extending from a distal end of the first portion, the second portion being more flexible than the first portion and configured to be inserted into a fistula vein of a patient. A needle is removably disposed in the cannula and configured to be displaced axially within the cannula.

### TECHNICAL FIELD

The present disclosure relates generally to cannulas for cannulating a fistula vein of a patient.

### BACKGROUND

Patients suffering from renal damage or failure typically undergo hemodialysis on a regular basis for removing toxins that accumulate in the blood. Hemodialysis involves filtering of wastes, toxins and water from blood, a function that is normally performed by the kidneys. Hemodialysis helps control blood pressure and maintain balance of important minerals in the blood, such as sodium, potassium, and calcium. An arteriovenous (AV) fistula may be grafted into the body (e.g., forearm or upper arm) of the patient to allow a cannula to be inserted for drawing blood out of the patient and delivering blood back to the patient. An AV fistula is a connection of an artery to a vein of the patient, generally performed by a vascular surgeon. The AV fistula causes extra pressure and extra blood to flow into the vein, causing an increase in size and strength of the vein. The larger vein (referred to herein as “fistula vein”) provides easy and reliable access for a cannula to be inserted therein. A cannula can be inserted repeatedly into such a fistula vein over numerous hemodialysis sessions. This would not be possible with normal veins because repeated needle insertions can collapse the vein when suction is applied in the cannula for drawing blood out of the vein. Traditionally, hemodialysis fistulas were formed using an invasive surgical procedure. Recently, percutaneous fistula vein formation procedures have been used to form a fistula vein with the proximal radial artery in the forearm via a minimally invasive procedure.

Patients (e.g., end stage renal failure patients) are generally required to regularly visit a medical provider or a hospital to undergo dialysis. This is inconvenient for such patients because they may also suffer from limited mobility and other health issues, and also increases their financial burden. Home dialysis machines are now available that allow patients to perform dialysis in the comfort of their homes. However, in the absence of a medical provider, the patients have to insert a cannula into the fistula vein themselves or a home caregiver. Currently available cannulas are difficult to insert into fistula veins and can also damage the fistula vein if not inserted properly. Moreover, the patients may have to insert two cannulas into the fistula vein, one for drawing blood and one for returning cleaned blood, making the process even more difficult.

### SUMMARY

Embodiments described herein relate generally to cannula assemblies and methods of using cannula assemblies for cannulating a fistula vein of a patient. In particular, embodiments described herein relate generally to cannula assemblies that include cannulas including a higher flexibility portion that is configured to be inserted into the fistula vein, and to cannulas including a fluid receiving channel and a fluid delivery channel integrated into a single cannula, and methods of using such cannula assemblies.

In some embodiments, a cannula assembly comprises a cannula comprising: a first portion, and a second portion extending from a distal end of the first portion, the second portion being more flexible than the first portion, and configured to be inserted into a fistula vein of a patient; and a needle removably disposed in the cannula and configured to be displaced axially within the cannula.

In some embodiments, a cannula assembly comprises a cannula comprising: a first channel, a second channel disposed adjacent to the first channel and fluidly separated from the first channel by a wall, and a tip portion located at a distal end of the first channel and/or the second channel and configured to be inserted into a fistula vein of a patient, the first channel and the second channel merging into a single channel at the tip portion; and a needle removably disposed in the first channel and configured to be displaced axially through the first channel so as to be selectively extendable through the tip portion beyond the distal end of the cannula, wherein one of the first channel or the second channel is configured to deliver a fluid into the fistula vein of a patient, and the other of the first channel or the second channel is configured to receive a fluid from the fistula vein.

In some embodiments, a method for cannulating a fistula vein of a patient comprises: providing a cannula assembly comprising: a cannula comprising: a first portion, and a second portion extending from a distal end of the first portion, the second portion being more flexible than the first portion, and configured to be inserted into the fistula vein; and a needle removably disposed in the cannula and configured to be displaced axially within the cannula; pushing the needle through the first portion and the second portion until a tip of the needle extends beyond a distal end of the second portion; inserting the needle into the fistula vein until at least the distal end of the second portion of the cannula is inserted into the fistula vein; retracting the needle from the second portion and out of the first portion; inserting the second portion into the fistula vein up to a desired length; and delivering a fluid into, or drawing a fluid out of the fistula vein via the cannula.

In some embodiments, a method for cannulating a fistula vein of a patient comprises providing a cannula assembly, comprising: a cannula comprising: a first channel, a second channel disposed adjacent to the first channel and fluidly separated from the first channel by a wall, and a tip portion located at a distal end of the first channel and/or the second channel and configured to be inserted into the fistula vein, the first channel and the second channel merging into a single channel at the tip portion; and a needle removably disposed in the first channel and configured to be displaced axially through the first channel; pushing the needle through the first channel until a tip of the needle extends beyond the tip portion through the distal end of the cannula; inserting the needle into the fistula vein until at least the tip portion is inserted into the fistula vein; retracting the needle from the first channel and out of the cannula; inserting the cannula into the fistula vein up to a desired length; delivering a fluid into the fistula vein via one of the first channel or the second channel; and drawing a fluid out of the fistula vein via the other of the first channel or the second channel.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

The foregoing and other features of the present disclosure will become more fully apparent from the following description and appended claims, taken in conjunction with the accompanying drawings. Understanding that these drawings depict only several implementations in accordance with the disclosure and are, therefore, not to be considered limiting of its scope, the disclosure will be described with additional specificity and detail through use of the accompanying drawings.

FIG. 1 is a schematic cross-sectional side view of a cannula assembly, according to a first embodiment.

FIG. 2 is a schematic cross-sectional side view of a cannula assembly, according to a second embodiment.

FIG. 3 is a schematic cross-sectional side view of a cannula assembly, according to a third embodiment.

FIG. 4 is a schematic cross-sectional side view of a cannula assembly, according to a fourth embodiment.

FIG. 5 is a flow chart for a method for cannulating a fistula vein of a patient using the cannula assembly of FIG. 1, according to an embodiment.

FIG. 6 is a flow chart for a method for cannulating a fistula vein of a patient using the cannula assembly of FIG. 2, 3, or 4 according to another embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to cannula assemblies and methods of using cannula assemblies for cannulating a fistula vein of a patient. In particular, embodiments described herein relate generally to cannula assemblies that include cannulas including a higher flexibility portion that is configured to be inserted into the fistula vein, and to cannulas including a fluid receiving channel and a fluid delivery channel integrated into a single cannula, and methods of using such cannula assemblies.

Embodiments of the cannula assemblies described herein may provide one or more benefits including, for example: (1) providing a cannula including a flexible portion that can be easily manipulated for inserting into complex shaped fistula veins and traversing the tortious path of such cannulas; (2) reducing backpressure and increasing flow rate without increasing a luminal diameter of the cannula; (3) providing ease of use which allows a minimally trained patient to self-cannulate the patient's fistula vein in a home setting without requiring an expert medical professional; (4) enabling fluid (e.g., blood) to be delivered to the fistula vein and received from the fistula vein via a single cannula which allows the patient to only cannulate him/herself once for performing a hemodialysis session; and (5) reducing hospital visits, which reduces medical costs.

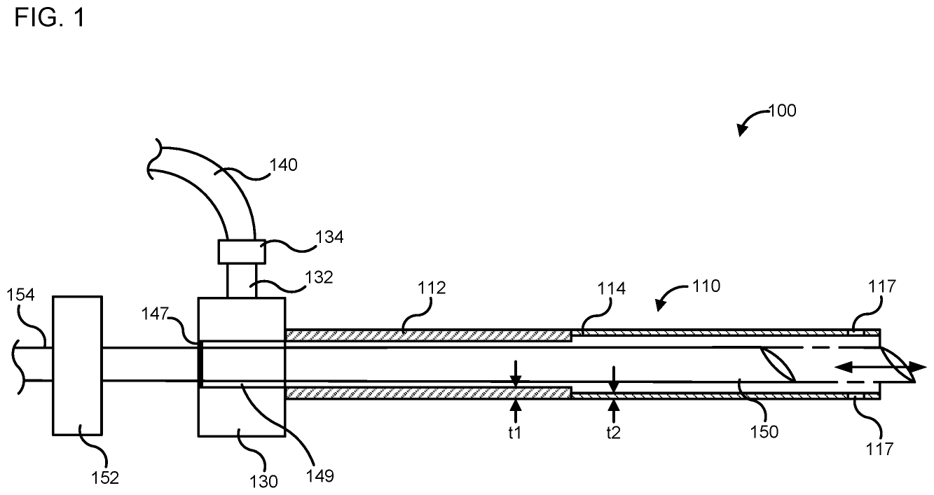

FIG. 1 is a schematic cross-sectional side view of a cannula assembly 100, according to an embodiment. The cannula assembly 100 includes a cannula 110, a hub 130, and a needle 150. The cannula 110 may be configured to be inserted into a fistula vein of a patient to perform hemodialysis, as described herein. It should be appreciated that while various embodiments of the cannula assemblies described herein are described in the context of hemodialysis for receiving blood from and/or delivering filtered blood to a fistula, in other embodiments, the cannula assemblies described herein can be used for cannulating a fistula vein, vein, or artery of a patient for delivering any fluid (e.g., blood, plasma, saline, drugs, platelets, etc.) or drawing/draining any fluid (e.g., blood, cerebrospinal fluid, pus, etc.) from a vein, artery, muscle, or skin of a patient.

The cannula 110 may have any suitable outer cross-sectional width (e.g., diameter). For example, the cannula 110 may be a 14 gauge cannula (2.1 mm outer diameter), a 16 gauge cannula (1.8 mm outer diameter), an 18 gauge cannula (1.3 mm outer diameter), a 20 gauge cannula (1.1 mm outer diameter), a 22 gauge cannula (0.9 mm outer diameter), a 24 gauge cannula (0.7 mm outer diameter), a 26 gauge cannula (0.5 mm outer diameter), or any other suitable outer diameter cannula.

The cannula 110 comprises a first portion 112, and a second portion 114 extending from a distal end of the first portion. The second portion 114 is more flexible than the first portion 112 and is configured to be inserted into the fistula vein of a patient. The higher flexibility of the second portion 114 allows the second portion 114 to easily flex which facilitates insertion of the second portion 114 into complex shaped fistula veins defining tortious flow paths. In some embodiments, the first portion 112 and the second portion 114 may be formed monolithically. In other embodiments, the second portion 114 may be formed separately from the first portion 112 and a proximal end of the second portion 114 may be coupled to the distal end of the first portion 112 (e.g., via a clamp, an adhesive, or fusion bonded thereto).

As used herein, the term “distal” when used in conjunction with a location of the cannula 110 or any other cannula described herein refers to location that is distant from a user and proximate to the fistula vein. Similarly, the term “proximal” when used in conjunction with a location of the cannula 110 or any other cannula described herein refers to location that is located proximate to a user and distant from the fistula.

In some embodiments as shown in FIG. 1, a portion of a wall of the cannula 110 forming the first portion 112 has a first thickness t1 (e.g., in a range of about 0.05 mm to about 0.5 mm, inclusive), and a portion of the wall of the cannula 110 forming the second portion 114 may have a second thickness t2 that is less than the first thickness. For example, the second thickness may be 0.75 times or less than the first thickness, or 0.5 times or less than the first thickness, or 0.25 times or less than the first thickness. The smaller thickness t2, for example, in a range of about 0.01 mm to about 0.4 mm (e.g., 0.01 mm to 0.05 mm, 0.05 mm to 0.1 mm, 0.1 mm to 0.2 mm, 0.2 mm to 0.3 mm, and 0.3 mm to 0.4 mm, inclusive) of the second portion 114 relative to the first portion 112 allows the second portion 114 to have a higher flexibility (e.g., bendability) than the first portion. In such embodiments, the first portion 112 and the second portion 114 may be formed from the same material such as, for example, polytetrafluoroethylene (PTFE), plastics (e.g., polyethylene, low density polyethylene, high density polyethylene), polyurethane, silicone, or any other suitable material. The cannula 110 may have a uniform outer diameter, such that the smaller second wall thickness t2 of the causes the second portion 114 to have a larger inner diameter than the first portion 112, which may facilitate fluid (e.g., blood) communication into or out of the fistula.

In other embodiments, the first portion 112 may be formed from a first material and the second portion 114 may be formed from a second material that is more flexible than the first material. For example, the first material may have a first elastic modulus (e.g., in a range of 0.38 GPa to 2.25 GPa) and the second material may have a second elastic modulus that is smaller than the first elastic modulus which causes the second portion 114 to be more flexible than the first portion 112. For example, the first portion 112 may be formed from harder and less flexible plastic, polytetrafluoroethylene (PTFE), polyurethane, silicone, or any other material having a first elastic modulus and the second portion 114 may be formed from a softer and more flexible plastic, PTFE, polyurethane, silicone, or any other material that has a second elastic modulus less than the first elastic modulus. In this embodiment, the second thickness t2 may be substantially equal to the first thickness t1 (e.g., within +10% thereof).

In some embodiments, one or more apertures 117 extend radially through a wall of the second portion 114 at a distal end of the second portion 114, which is configured to be inserted into the fistula vein. The one or more apertures 117 provide additional locations for the fluid (e.g., blood) to exit the distal end of the cannula 110, or be drawn into the cannula 110. The one or more apertures 117 may beneficially reduce back pressure and/or increase flow rate through the cannula 110.

The cannula assembly 100 may also include a hub 130 coupled to a proximal end of the first portion 112 of the cannula 110 opposite the second portion 114. The hub 130 defines a fluid conduit 132 to which a tube 140 configured to deliver fluid (e.g., blood) into, or receive fluid (e.g., blood) from the cannula 110 is coupled. A securing member 134, for example, a nut, clamp, or a luer lock, may be used to couple the tube 140 to the fluid conduit 132. The needle 150 extends through the hub 130 and is axially displaceable through the hub 130. For example, the hub 130 may define a channel 149 through which the needle 150 is disposed. In some embodiments, the hub 130 may be transparent or translucent, for example, to allow the patient or caregiver to observe the flow of the fluid through the hub 130.

In some embodiments, a sealing valve 147 (e.g., a septum) may be disposed in the hub 130 at a proximal end of the channel 149. The needle 150 may be inserted through the sealing valve 147 into the channel 149 and therethrough into the cannula 110. The sealing valve 147 is configured to reseal once the needle 150 is removed from the cannula 110 and the hub 130 to prevent fluid leakage.

The needle 150 is removably disposed in the cannula 110 and configured to be displaced axially within the cannula 110. A needle hub 152 is coupled to a proximal end of needle 150, and may be configured to be engaged by a user (e.g., the patient or caregiver) to displace the needle 150 within the cannula 110. In some embodiments, a fluid conduit 154 may be coupled to the needle 150, and configured to provide a fluid through the needle 150. The needle 150 may be used to pierce the fistula vein to allow insertion of the second portion 114 of the cannula 110 therewith into the fistula vein. For example, the needle 150 may be pushed through the cannula 110 until a tip of the needle 150 extends beyond the distal end of the cannula 110 and the needle 150 cannot be pushed any further through the cannula 110 due to the needle hub 152 contacting the hub 130. The patient or caregiver may then insert the tip of the needle 150 into the fistula vein and continue insertion until at least the distal end of the second portion 114 is inserted into the opening formed in the fistula vein by the tip of the needle 150. The patient or caregiver may then retract the needle 150 out of the cannula 110. The patient or caregiver may further insert the second portion 114 into the fistula vein up to a desired length and then secure the cannula 110 in place, for example, via medical tape.

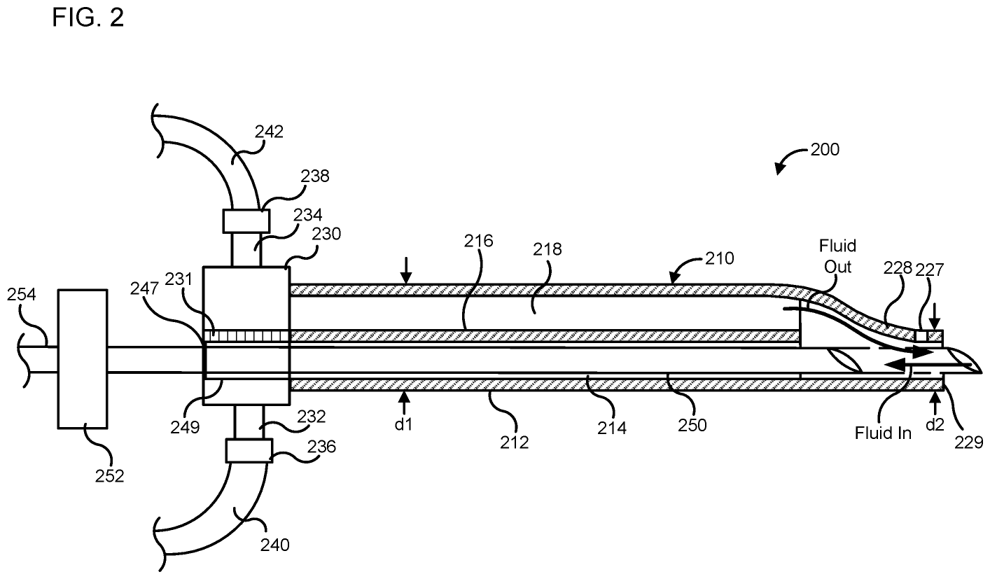

FIG. 2 is a schematic cross-sectional side view of a cannula assembly 200, according to another embodiment. The cannula assembly 200 comprises a cannula 210, a hub 230, and a needle 250. The cannula 210 may be configured to be inserted into a fistula vein of a patient to perform hemodialysis or any other fluid transport. The cannula assembly 200 is configured to receive a fluid (e.g., blood) from the fistula vein as well as deliver a fluid (e.g., blood) to the fistula vein.

The cannula 210 includes a first channel 214, and a second channel 218 disposed adjacent to the first channel 214 and fluidly separated from the first channel 214 by a wall 216. As shown in FIG. 2, the first channel 214 is formed between an outer wall 212 of the cannula 210 and the wall 216, and the second channel 218 is also formed between the outer wall 212 and the wall 216 such that the wall 216 fluidly separates the first channel 214 from the second channel 218.

A tip portion 228 is located at a distal end of the cannula 210 and is configured to be inserted into the fistula vein. The first channel 214 and the second channel 218 merge into a single channel at the tip portion 228. As shown in FIG. 2, the wall 216 extends into the cannula 210 up to the tip portion 228 and ends before the tip portion 228 such that first and second channels 214 and 218 merge together into a single channel at the tip portion 228.

The cannula 210 has a first cross-sectional width d1 (e.g., in a range of about 0.5 mm to about 2.2 mm) at locations where the first channel 214 is fluidly separated from the second channel 218. The tip portion 228 has a second cross-sectional width d2 at distal end 229 thereof that is smaller than the first cross-sectional width d1 of the cannula 210 (e.g., 0.75 times, 0.5 times, or 0.25 times the first cross-sectional width d1, inclusive). For example, the outer wall 212 of the cannula 210 tapers towards the first channel 214 at the tip portion 228 such that that the second cross-sectional width d2 of the tip portion 228 at a distal end 229 of the cannula 210 is smaller than the first cross-sectional width d1. In some embodiments, the second cross-sectional width d2 is substantially the same as a cross-sectional width of the first channel 214 (e.g., within +10% of the cross-sectional width of the first channel 214).

The cannula 210 may have any suitable first cross-sectional width d1. For example, the cannula 210 may be a 14 gauge cannula (2.1 mm outer diameter), a 16 gauge cannula (1.8 mm outer diameter), an 18 gauge cannula (1.3 mm outer diameter), a 20 gauge cannula (1.1 mm outer diameter), a 22 gauge cannula (0.9 mm outer diameter), a 24 gauge cannula (0.7 mm outer diameter), a 26 gauge cannula (0.5 mm outer diameter), or any other suitable outer diameter cannula. The cannula 210 may be formed from any suitable material, for example, polytetrafluoroethylene (PTFE), plastics (e.g., polyethylene, low density polyethylene, high density polyethylene), polyurethane, silicone, etc.

The tip portion 228 is configured to be inserted into the fistula. In some embodiments, one or more apertures 227 may extend radially through the outer wall 212 of the cannula 210 at the distal end 229, for example, to increase flow rate and/or reduce back pressure. In some embodiments, only one aperture 227 may extend through the outer wall 212 at the tip portion 228 at a location of the outer wall 212 that is distal to the second channel 218 through which fluid is delivered into the fistula vein. This may overcome back pressure due to fluid entering from the fistula vein into the cannula 210 through the tip portion 228 as well as the aperture 227.

One of the first channel 214 or the second channel 218 is configured to deliver fluid to the fistula vein, and the other of the first channel 214 or the second channel 218 is configured to receive fluid from the fistula vein. For example, the first channel 214 may be configured to receive fluid (e.g., blood) from the fistula vein, and the second channel 218 may be configured to deliver fluid (e.g., filtered blood) to the fistula vein, as shown in FIG. 2.

The hub 230 is coupled to a proximal end of the cannula 210 opposite the tip portion 228. The hub 230 defines a first fluid conduit 232 that is in fluid communication with the first channel 214, and a second fluid conduit 234 that is in fluid communication with the second channel 218. The first fluid conduit 232 and the second fluid conduit 234 may be fluidly isolated from each other via a hub wall 231. One of the first fluid conduit 232 or the second fluid conduit 234 may serve as a fluid inlet for delivering fluid to the corresponding one of the first channel or the second channel, and the other of the first fluid conduit 232 or the second fluid conduit 234 may serve as fluid outlet for receiving a fluid from the other of the first channel 214 or the second channel 218. For example, as shown in FIG. 2, the first fluid conduit 232 receives fluid (e.g., blood) from the first channel 214, and the second fluid conduit 234 delivers fluid (e.g., filtered blood) to the second channel 218. Alternatively, the first fluid conduit 232 may receive fluid (e.g., blood) from the first channel 214, and the second fluid conduit 234 may deliver fluid (e.g., filtered blood) to the second channel 218. In some embodiments, the hub 230 may be transparent or translucent, for example, to allow the patient or caregiver to observe the flow of the fluid through the hub 230.

Securing members 236 and 238, for example, nuts, clamps, or luer locks, may be used to couple tubes 240 and 242 to the first fluid conduit 232 and the second fluid conduit 234, respectively. The needle 250 is disposed through the hub 230 and axially displaceable therethrough. For example, the hub 230 may define a channel 249 that is axially aligned with the first channel 214, through which the needle 250 is disposed. In some embodiments, a sealing valve 247 (e.g., a septum) may be disposed in the hub 230 at a proximal end of the channel 249. The needle 250 may be inserted through the sealing valve 247 into the channel 249 and therethrough into the first channel 214. The sealing valve 247 is configured to reseal once the needle 250 is removed from the cannula 210 and the hub 230 to prevent fluid leakage.

The needle 250 is removably disposed in the first channel 214 and configured to be displaced axially through the first channel 214 so as to be selectively extendable through the tip portion 228 beyond the distal end of the cannula 210. A needle hub 252 is coupled to a proximal end of needle 250, and may be configured to be engaged by a user (e.g., the patient or caregiver) to displace the needle 250 within the first channel 214 of cannula 210. In some embodiments, a fluid conduit 254 may be coupled to the needle 250, and configured to provide a fluid through the needle 250. The needle 250 may be substantially similar to the needle 150 and therefore, not described in further detail herein.

The user may push the needle 250 through the first channel 214 of the cannula 210 until a tip of the needle 250 extends beyond the distal end of the cannula 210 and the needle 250 cannot be pushed any further through the cannula 210 due to the needle hub 252 contacting the hub 230. The patient or caregiver may insert the needle 250 into the fistula vein until the tip portion 228 is also inserted along with the needle 250 into the fistula vein. The patient or caregiver can then retract the needle 250 from the first channel 214 and out of the cannula 210, and then insert the cannula 210 up to desired length into the fistula vein. The cannula 210 can be used to deliver fluid to or draw fluid from the fistula vein, for example, draw blood from the fistula vein and deliver filtered blood to the fistula vein to perform hemodialysis without the use of two cannulas. In some embodiments, the fluid (e.g., blood) may be received from the fistula vein and delivered to the fistula vein simultaneously. In other embodiments, the fluid (e.g., unfiltered blood) may first be drawn from the fistula vein via the first channel 214 for a first time period and thereafter the fluid (e.g., filtered blood) may be delivered to the fistula vein for a second time period, and the process repeated. In other words, fluid drawing and fluid delivery pulses may be applied to sequentially draw fluid and deliver fluid to the fistula vein.

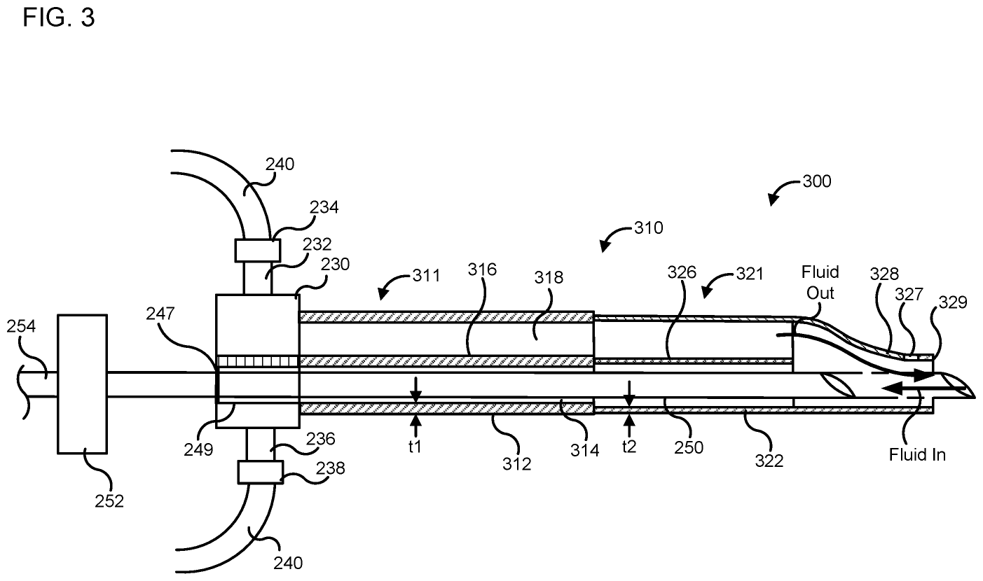

FIG. 3 is a schematic cross-sectional side view of a cannula assembly 300, according to another embodiment. The cannula assembly 300 comprises a cannula 310, the hub 230, and the needle 250. The cannula 310 may be configured to be inserted into a fistula vein of a patient to perform hemodialysis or any other fluid transport. The cannula assembly 300 is configured to receive a fluid (e.g., blood) from the fistula vein, and deliver fluid (e.g., filtered blood) to the fistula vein.

The cannula 310 includes a first channel 314, and a second channel 318 disposed adjacent to the first channel 314 and fluidly separated from the first channel 314 by a wall 316. As shown in FIG. 3, the first channel 314 is formed between on outer wall 312 of the cannula 310 and the wall 316, and the second channel 318 is also formed between the outer wall 312 and the all 316 such that the wall 316 fluidly separates the first and second channels 314 and 318. A tip portion 328 is located at a distal end 329 of the cannula 310 and is configured to be inserted into the fistula vein. The first channel 314 and the second channel 318 merge into a single channel at the tip portion 328.

The tip portion 328 is configured to be inserted into the fistula vein. In some embodiments, one or more apertures 327 may extend radially through the outer wall 212 of the cannula 310 at the distal end 329, for example, to increase flow rate and/or reduce back pressure. One of the first channel 314 or the second channel 318 is configured to deliver fluid to the fistula vein, and the other of the first channel 314 or the second channel 318 is configured to receive fluid from the fistula vein. For example, the first channel 314 may be configured to receive fluid (e.g., blood) from the fistula vein, and the second channel 318 may be configured to deliver fluid (e.g., filtered blood) to the fistula vein, as shown in FIG. 3.

Different from the cannula 210, the cannula 310 includes a first portion 311 including a first segment of the first channel 314 and the second channel 318, and a second portion 321 extending from a distal end of the first portion 311. The second portion 321 includes a second segment of the first channel 314 and the second channel 318, as well as the tip portion 328 which is located proximate to the fistula vein. The second portion 321 is more flexible than the first portion 311 and is configured to be inserted into the fistula vein.

In some embodiments as shown in FIG. 3, a portion of the outer wall 312 of the cannula 310 and, in some embodiments, the wall 316 forming the first portion 311 has a first thickness t1 (e.g., in a range of about 0.05 mm to about 0.5 mm, inclusive). Moreover, a portion of the outer wall 312 of the cannula 310 and, in some embodiments, the wall 316, forming the second portion 321 may have a second thickness t2 that is less than the first thickness e.g., 0.75 times or less than the first thickness, or 0.5 times or less than the first thickness, or 0.25 times or less than the first thickness. The smaller thickness t2, for example, in a range of about 0.01 mm to about 0.4 mm (e.g., 0.01 mm to 0.05 mm, 0.05 mm to 0.1 mm, 0.1 mm to 0.2 mm, 0.2 mm to 0.3 mm, and 0.3 mm to 0.4 mm, inclusive) of the second portion 321 relative to the first portion 311 allows the second portion 321 to have a higher flexibility (e.g., bendability) than the first portion 311. In such embodiments, the first portion 311 and the second portion 321 may be formed from the same material such as, for example, polytetrafluoroethylene (PTFE), plastics (e.g., polyethylene, low density polyethylene, high density polyethylene), polyurethane, silicone, or any other suitable material. The cannula 310 may have a uniform outer diameter, such that the smaller second wall thickness t2 of the second portion 321 causes the second portion 321 to have a larger inner diameter than the first portion 311, which may facilitate fluid (e.g., blood) communication into and out of the fistula.

In other embodiments, the first portion 311 may be formed from a first material and the second portion 321 may be formed from a second material that is more flexible than the first material. For example, the first material may have a first elastic modulus (e.g., in a range of 0.38 GPa to 2.25 GPa), and the second material may have a second elastic modulus that is smaller than the first elastic modulus causing the second portion 321 to be more flexible than the first portion 311. For example, the first portion 311 may be formed from harder and less flexible plastic, polytetrafluoroethylene (PTFE), polyurethane, silicone, or any other material having a first elastic modulus and the second portion 114 may be formed from a softer and more flexible plastic, PTFE, polyurethane, silicone, or any other material that has a second elastic modulus less than the first elastic modulus. In such embodiment, the second thickness t2 may be substantially equal to the first thickness t1 (e.g., within +10% thereof).

The needle 250 is removably disposed in the first channel 314 and configured to be displaced axially through the first channel 314 so as to be selectively extendable through the tip portion 328 beyond the distal end of the cannula 310, as described with respect to the cannula assembly 200. The hub 230 is coupled to a proximal end of the cannula 210 opposite the tip portion 228, as already described in detail with respect to the cannula assembly 200.

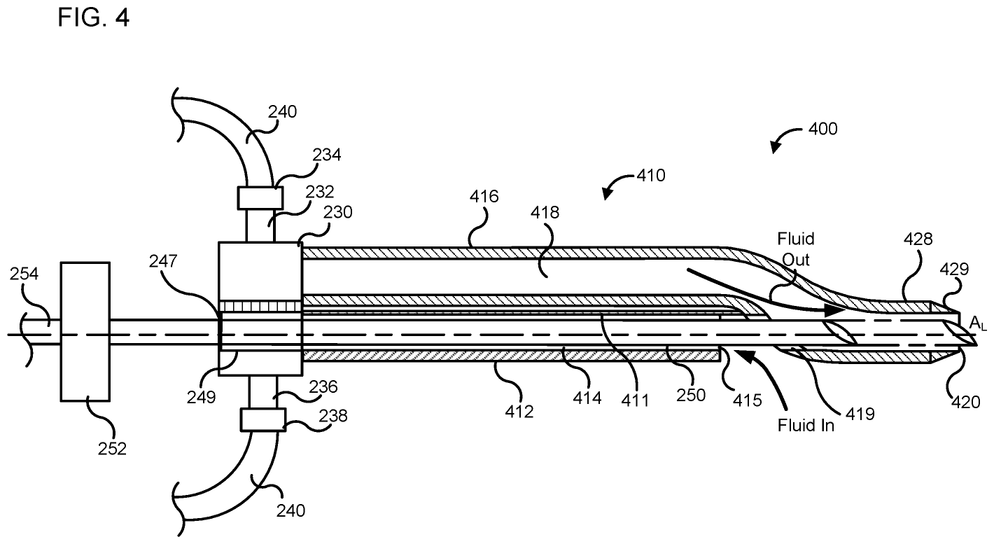

FIG. 4 is a schematic cross-sectional side view of a cannula assembly 400, according to yet another embodiment. The cannula assembly 400 comprises a cannula 410, the hub 230, and the needle 250. The cannula 410 may be configured to be inserted into a fistula vein of a patient to perform hemodialysis or any other fluid transport. The cannula assembly 200 is configured to receive a fluid (e.g., blood) from the fistula vein as well as deliver a fluid (e.g., blood) to the fistula vein.

The cannula 210 includes a first tube 412 defining a first channel 414. In some embodiments, the first channel 414 may be configured to receive a fluid (e.g., unfiltered blood) from the fistula vein. The cannula 210 also includes a second tube 416 disposed adjacent to the second tube such that an outer wall of the second tube 416 is coupled to an outer wall of the first tube 412. In some embodiments, the first tube 412 and the second tube 416 may be monolithically formed. In some embodiments, a thin separating layer 411, for example, a septum may be disposed between the first tube 412 and the second tube 416 to fluidly separate the first tube 412 and the second tube 416, as shown in FIG. 4. The second tube 416 defines a second channel 418. In some embodiments, the second channel 418 may be configured to deliver fluid (e.g., filtered blood) to the fistula vein, as shown in FIG. 4. In other embodiments, the first channel 414 may be configured to deliver fluid to the fistula vein and the second channel 418 may be configured to receive fluid from the filtered vein.

The second channel 418 is longer than the first channel 414 and extends beyond a tip of the first channel 414. The second channel 418 includes a tip portion 428 located a distal end of the second channel 418 and configured to be inserted into the fistula vein. Expanding further, a distal end of the second channel 418 extends beyond the first channel 414 and curves towards a longitudinal axis A_{L} defined by the first channel 414 such that at least a segment of the tip portion 428 is axially aligned with the longitudinal axis A_{L}. In some embodiments, a cross-sectional width (e.g., diameter) of a distal end of the tip portion 428 may be substantially equal to (e.g., within +10%) of a cross-sectional width (e.g., diameter) of the first channel 414.

The second channel 418 defines a second channel first outlet 419 that is located proximate to and is axially aligned with a first channel inlet 415, and a second channel second outlet 420 axially aligned with and located distal from the first channel inlet 415. The hub 230 is coupled to a proximal end of the cannula 410. The needle 250 is removably disposed in the first channel 414 and configured to be displaced axially through the first channel 414. A tip of the needle 250 extends through the first channel inlet 415, and through the tip portion 428 via the second channel first outlet 419 and the second channel second outlet 420. In some embodiments, a distal end 429 of the tip portion 428 may be tapered so as to form a sharp end that may facilitate insertion of the tip portion 428 into the fistula vein. In various embodiments, a distal end of any of the cannulas described herein (e.g., the cannula 110, 210, 310) may be tapered to form a sharp tip.

For example, the patient or caregiver may engage the needle hub 252 to displace the needle 250 through the first channel 414 and the tip portion 428 defined by the second channel 418. The user may push the needle 250 through the first channel 414 and the tip portion 428 until the tip of the needle 250 extends beyond a distal end of the tip portion 428 and the needle 250 cannot be pushed any further through the first channel 414 due to the needle hub 252 contacting the hub 230.

The patient or caregiver may insert the needle 250 into the fistula vein until the tip portion 428 is also inserted along with the needle 250 into the fistula vein. The patient or caregiver can then retract the needle 250 from the tip portion 428, the first channel 414 and out of the first channel 414. The cannula 410 is then inserted up to desired length into the fistula vein. The cannula 210 can be used to deliver fluid to or draw fluid from the fistula vein, for example, draw blood from the fistula vein via the first channel inlet 414, and deliver filtered blood to the fistula vein via the second channel first outlet 419 and the second channel second outlet 420.

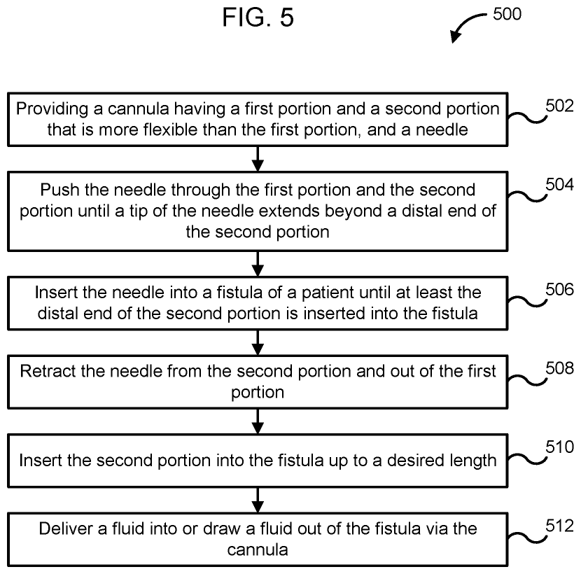

FIG. 5 is a flow chart for a method 500 for cannulating a fistula vein of a patient using the cannula assembly 100, according to an embodiment. The fistula vein may be cannulated, for example, to perform hemodialysis on the patient. The method 500 includes providing a cannula assembly (e.g., the cannula assembly 100), at 502. The cannula assembly comprises a cannula (e.g., the cannula 110), including a first portion (e.g., the first portion 112) and a second portion (e.g., the second portion 114) extending from the first portion. The second portion is more flexible than the first portion and is configured to be inserted into the fistula vein.

In some embodiments, a wall of the cannula forming the first portion has a first thickness, and a wall of the cannula forming the second portion has a second thickness that is less than the first thickness, as previously described herein. In some embodiments, the first portion of the cannula is formed from a first material, and the second portion of the cannula is formed from a second material that is more flexible than the first material. In some embodiments, at least one aperture (e.g., the aperture 117) extends radially through a wall of the second portion proximate to a distal end of the second portion, which is configured to be inserted into the fistula vein. A needle (e.g., the needle 150) is removably disposed in the cannula and configured to be displaced axially within the cannula. In some embodiments, a hub (e.g., the hub 130) is coupled to a proximal end of the cannula, as previously described herein.

At 504, the needle is pushed through the first portion and the second portion until a tip of the needle extends beyond a distal end of the second portion. At 506, the needle is retracted from the second portion and out of the first portion. At 508, the second portion of the cannula is inserted into the fistula vein up to a desired length. At 510, a fluid is delivered into, or drawn out of the fistula vein via the cannula depending on whether the user intends to use the cannula as a fluid drawing cannula or a fluid delivery cannula. For example, blood may be drawn out of the fistula vein via the cannula, and a separate cannula may be used to deliver filtered blood into the fistula vein or vice versa.

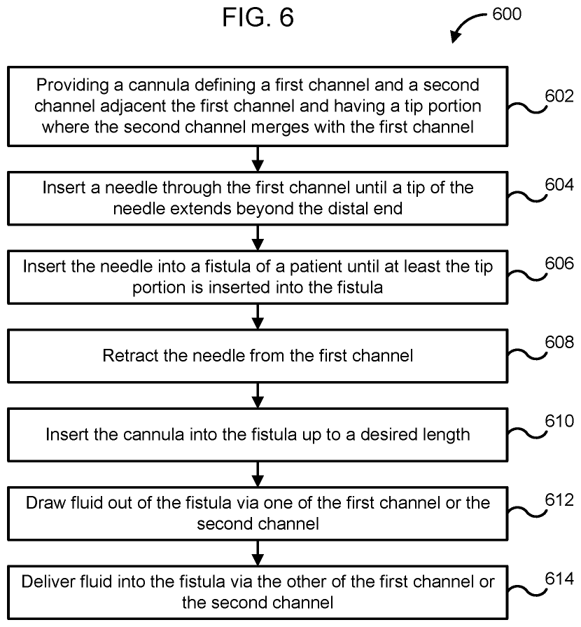

FIG. 6 is a flow chart for a method 600 for cannulating a fistula vein of a patient using the cannula assembly 200, 300, or 400, according to an embodiment. The fistula vein may be cannulated, for example, to perform hemodialysis on the patient. The method 600 includes providing a cannula assembly (e.g., the cannula assembly 200, 300, 400), at 602. The cannula assembly includes a cannula (e.g., the cannula 210, 310, 410) including a first channel (e.g., the first channel 214, 314, 414), and a second channel (e.g., the second channel 218, 318, 418) disposed adjacent to the first channel, for example, fluidly separated from the first channel by a wall (e.g., the wall 216, 316). A tip portion (e.g., the tip portion 228, 328, 428) is located at a distal end of the cannula and is configured to be inserted into the fistula. The first channel and the second channel may be fluidly coupled into a single channel at the tip portion, or the tip portion (e.g., the tip portion 428) may be defined by one of the first channel or the second channel (e.g., the second channel 418), as previously described herein.

The tip portion of the cannula has a cross-sectional width that is smaller than a cross-sectional width of the cannula at locations of the cannula where the first channel is separated from the second channel. For example, an outer wall of the cannula tapers towards the first channel at the tip portion such that a cross-sectional width of the tip portion at a distal end thereof is substantially the same as a cross-sectional width of the first channel. In some embodiments, at least one aperture (e.g., the aperture 217, 317) extends radially through a wall of the tip portion, as previously described herein. In some embodiments, the cannula includes a first portion (e.g., the first portion 311) including a first segment of the first channel and the second channel, and a second portion (e.g., the second portion 321) extending from a distal end of the first portion. The second portion includes a second segment of the first channel and the second channel. The second portion is more flexible than the first portion and is configured to be inserted into the fistula vein, as previously described herein. A needle (e.g., the needle 250) is removably disposed in the first channel and configured to be displaced axially through the first channel. A hub (e.g., the hub 230) may be coupled to a proximal end of the cannula. In other embodiments, the tip portion (e.g., the tip portion 428) may be defined by a distal end of a second channel (e.g., the second channel 418) that is coupled to a first channel (e.g., the first channel 414). In such embodiments, the needle is removably displaced in the first channel and the tip portion defined by the second channel.

At 604, the needle is pushed through the second channel until a tip of the needle extends beyond the tip portion through the distal end of the cannula. At 606, the needle is inserted into the fistula vein until at least the tip portion is inserted into the fistula vein. At 608, the needle is retracted from the first channel and out of the cannula. At 610, the cannula is inserted into the fistula vein up to a desired length. At 612, a fluid (e.g., blood) is drawn out of the fistula vein via one of the first channel or the second channel of the cannula (e.g., via the first channel 214, 314, 414). At 614, a fluid (e.g., blood) is delivered to the fistula vein via the other of the first channel or the second channel (e.g., via the second channel 218, 318, 418). For example, the cannula may be fluidly coupled to a hemodialysis machine (e.g., via the hub 230) for filtering blood of the patient. The drawing and delivery of the fluid may be performed simultaneously or sequentially as previously described herein.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

What is claimed is:

1. A cannula assembly, comprising:
a cannula comprising:
a first portion, and
a second portion extending from a distal end of the first portion, the second portion being more flexible than the first portion and configured to be inserted into a fistula vein of a patient; and
a needle removably disposed in the cannula and configured to be displaced axially within the cannula.

2. The cannula assembly of claim 1, wherein a wall of the cannula has a first thickness at the first portion and a second thickness at the second portion, the second thickness being less than the first thickness.

3. The cannula assembly of claim 1, wherein the first portion of the cannula is formed from a first material and the second portion of the cannula is formed from a second material, the second material being more flexible than the first material.

4. The cannula assembly of claim 1, wherein one or more apertures extend radially through a wall of the second portion at a distal end of the second portion.

5. The cannula assembly of claim 1, further comprising:
a hub coupled to a proximal end of the first portion of the cannula, the hub defining a fluid conduit configured to deliver a fluid into or receive a fluid from the cannula,
wherein the needle extends through the hub and is axially displaceable through the hub.

6. A cannula assembly comprising:
a cannula comprising:
a first channel,
a second channel disposed adjacent to the first channel and fluidly separated from the first channel by a wall, and
a tip portion located at a distal end of the first channel and/or the second channel and configured to be inserted into a fistula vein of a patient, the first channel and the second channel merging into a single channel at the tip portion; and
a needle removably disposed in the first channel and configured to be displaced axially through the first channel so as to be selectively extendable through the tip portion beyond the distal end of the cannula,
wherein one of the first channel or the second channel is configured to deliver a fluid into the fistula vein, and the other of the first channel or the second channel is configured to receive a fluid from the fistula vein.

7. The cannula assembly of claim 6, wherein a cross-sectional width of the tip portion of the cannula is smaller than a cross-sectional width of the cannula at locations where the first channel is separated from the second channel by the wall.

8. The cannula assembly of claim 7, wherein an outer wall of the cannula tapers towards the first channel at the tip portion such that a cross-sectional width of the tip portion at the distal end thereof is substantially the same as a cross-sectional width of the first channel.

9. The cannula assembly of claim 6, wherein a distal end of the second channel curves towards a longitudinal axis defined by the first channel to form the tip portion, at least a segment of the tip portion being axially aligned with the longitudinal axis of the first channel,
wherein the needle is extendable through a first channel inlet and through the tip portion via a second channel first outlet defined in the tip portion proximate to the first channel inlet, and the second channel second outlet located distal from the first channel inlet.

10. The cannula assembly of claim 6, wherein one or more apertures extend radially through a wall of the tip portion at the distal end thereof.

11. The cannula assembly of claim 6, wherein the cannula comprises:
a first portion comprising a first segment of the first channel and the second channel, and
a second portion extending from a distal end of the first portion, the second portion comprising a second segment of the first channel and the second channel, and the tip portion, the second portion being more flexible than the first portion and configured to be inserted into the fistula.

12. The cannula assembly of claim 6, further comprising:
a hub coupled to a proximal end of the cannula, the hub defining a fluid inlet for delivering the fluid to one of the first channel or the second channel, and a fluid outlet for receiving the fluid from the other of the first channel or the second channel,
wherein the needle extends through the hub and is axially displaceable through the hub.

13. A method for cannulating a fistula vein of a patient, comprising:
providing a cannula assembly, comprising:
a cannula comprising:
a first portion, and
a second portion extending from a distal end of the first portion, the second portion being more flexible than the first portion and configured to be inserted into the fistula vein; and
a needle removably disposed in the cannula and configured to be displaced axially within the cannula;
pushing the needle through the first portion and the second portion until a tip of the needle extends beyond a distal end of the second portion;
inserting the needle into the fistula vein until at least the distal end of the second portion of the cannula is inserted into the fistula vein;
retracting the needle from the second portion and out of the first portion;
inserting the second portion into the fistula vein up to a desired length; and
delivering a fluid into, or drawing a fluid out of the fistula vein via the cannula.

14. The method of claim 13, wherein a wall of the cannula has a first thickness at the first portion and a second thickness at the second portion, the second thickness being less than the first thickness.

15. The method of claim 13, wherein the first portion of the cannula is formed from a first material and the second portion of the cannula is formed from a second material, the second material being more flexible than the first material.

16. The method of claim 13, wherein one or more apertures extend radially through a wall of the second portion at the distal end of the second portion.

17. A method for cannulating a fistula vein of a patient, comprising:
providing a cannula assembly, comprising:
a cannula comprising:
a first channel,
a second channel disposed adjacent to the first channel and fluidly separated from the first channel by a wall, and
a tip portion located at a distal end of the first channel and/or the second channel and configured to be inserted into the fistula vein, the first channel and the second channel merging into a single channel at the tip portion; and
a needle removably disposed in the first channel and configured to be displaced axially through the first channel;
pushing the needle through the second channel until a tip of the needle extends beyond the tip portion through the distal end of the cannula;
inserting the needle into the fistula vein until at least the tip portion is inserted into the fistula;
retracting the needle from the first channel and out of the cannula;
inserting the cannula into the fistula vein up to a desired length;
delivering a fluid into the fistula vein via one of the first channel or the second channel; and
drawing a fluid out of the fistula vein via the other of the first channel or the second channel.

18. The method of claim 17, wherein a cross-sectional width of the tip portion of the cannula is smaller than a cross-sectional width of the cannula at locations of the cannula where the first channel is separated from the second channel by the wall.

19. The method of claim 18, wherein an outer wall of the cannula tapers towards the first channel at the tip portion such that a cross-sectional width of the tip portion at the distal end thereof is substantially the same as a cross-sectional width of the first channel.

20. The method of claim 17, wherein a distal end of the second channel curves towards a longitudinal axis defined by the first channel to form the tip portion, at least a segment of the tip portion being axially aligned with the longitudinal axis of the first channel,
wherein the needle is extendable through a first channel inlet and through the tip portion via a second channel first outlet defined in the tip portion proximate to the first channel inlet, and the second channel second outlet located distal from the first channel inlet.

21. The method of claim 16, wherein one or more apertures extend radially through an outer wall of the tip portion at the distal end thereof.

22. The method of claim 17, wherein the cannula comprises:
a first portion comprising a first segment of the first channel and the second channel; and
a second portion extending from a distal end of the first portion, the second portion comprising a second segment of the first channel and the second channel, and the tip portion, the second portion being more flexible than the first portion and configured to be inserted into the fistula vein.
